(19) United States
(12) Patent Application Publication
(10) Pub. No.: US 2026/0182968 A1
(43) Pub. Date: Jul. 2, 2026

(54) External Elastic Media (EEM)-Related Analytic Data Representation of Blood Vessel Images

(71) Applicant: ACIST Medical Systems, Inc., Eden Prairie, MN (US)

(72) Inventors: Jung Wook Suh, Pleasanton, CA (US); Enes Ibrahim Yazgan, Pleasanton, CA (US); Advit Prahlad Bhatt, Pleasanton, CA (US)

(73) Assignee: ACIST Medical Systems, Inc., Eden Prairie, MN (US)

(21) Appl. No.: 19/545,178

(22) Filed: Feb. 20, 2026

Related U.S. Application Data
(63) Continuation of application No. PCT/US2024/011179, filed on Jan. 11, 2024.
(60) Provisional application No. 63/603,741, filed on Nov. 29, 2023.

(51) Int. Cl. A61B 8/00; A61B 8/08; A61B 8/12

(52) U.S. Cl. CPC A61B 8/5223; A61B 8/085; A61B 8/12; A61B 8/463; A61B 8/465

(57) ABSTRACT

The embodiments presented herein relate to external elastic media (EEM)-related analytic data representation of intravascular images. In one embodiment, a non-transitory computer-readable medium is provided that stores program instructions. When executed by one or more processors, the program instructions cause the one or more processors to perform functions comprising: calculating EEM-related analytic data from each of a plurality of cross-sectional intravascular images; and causing a display of: a longitudinal view of the plurality of cross-sectional intravascular images at a selected cutting plane angle; and a longitudinal view of a visualization of the EEM-related analytic data that was calculated from each of the plurality of cross-sectional intravascular images. Other embodiments are provided.

### CROSS-REFERENCE TO RELATED APPLICATIONS

This application is a continuation of PCT Application No. PCT/US2024/011179, filed Jan. 11, 2024, which claims priority to U.S. Provisional Patent Application No. 63/603,741, filed Nov. 29, 2023, both of which are hereby incorporated by reference.

### BACKGROUND

An imaging procedure, such as an intravascular ultrasound image (IVUS) procedure, can be used to produce a series of images of a patient's vasculature, for instance, of the coronary artery lumen and coronary artery wall morphology. Automatic image segmentation can be used to simplify interpretation and measurement of key features of the generated ultrasound images, which can be used to assess coronary artery disease and to guide Percutaneous Coronary Interventions (e.g., the placement of a bare-metal or a drug-eluting stent, an Angioplasty procedure for squeezing the plaque and increasing the free cross-sectional area available to the blood flow, and an Atherectomy procedure for removal of a blood vessel blockage). Therefore, the availability of analytic data (such as plaque burden (PB), minimum lumen area (MLA), average lumen diameter, external elastic membrane (EEM) area), which can be processed from raw images collected by the imaging procedure, represents important information for assessing a disease severity of a patient's blood vessel under investigation, as well as for devising a treatment plan.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of an example medical diagnostic ultrasound imaging system of an embodiment.

FIG. 2 is a diagram illustrating an intravascular ultrasound image procedure of an embodiment.

FIG. 3 is a cross-sectional intravascular ultrasound image of an embodiment showing a lumen, plaque, and vessel wall.

FIG. 4A is a stack of cross-sectional intravascular ultrasound images of an embodiment.

FIG. 4B shows a cutting plane at a specific angle of the stack of cross-sectional intravascular ultrasound images of FIG. 4A.

FIG. 5A is a longitudinal view constructed from the stack of cross-sectional intravascular ultrasound images of FIG. 4A at a first cutting plane.

FIG. 5B is a longitudinal view constructed from the stack of cross-sectional intravascular ultrasound images of FIG. 4A at a second cutting plane.

FIG. 6 is an example cross-sectional intravascular ultrasound image with a denoted plaque burden based on automatically-segmented lumen and vessel wall boundaries.

FIG. 7 shows an example longitudinal representation of analytic information of an embodiment.

FIG. 8 shows an example longitudinal representation of analytic information of an embodiment.

FIG. 9 is a flowchart of a method of an embodiment for longitudinal analytic data representation of intravascular ultrasound images.

FIG. 10 is a screen shot of an acquire view of an embodiment.

FIG. 11 is a screen shot of an archive view of an embodiment.

FIG. 12 is a screen shot of a thumbnail preview mode of an embodiment.

FIG. 13 is a screen shot of an embodiment showing auto segmentation running.

FIG. 14 is a screen shot of a manual study annotation of an embodiment.

FIG. 15 is a screen shot of an embodiment in which a smart planning button is untoggled.

FIG. 16 is a screen shot of an embodiment showing a lumen and vessel wall longitudinal representation.

FIG. 17 is a screen shot of an embodiment showing longitudinal representation of average lumen, average vessel wall, and plaque burden across a loop.

FIG. 18 is a screen shot of an embodiment showing longitudinal representation with widest and narrowest lumen frames between selected bounds.

### DETAILED DESCRIPTION

### Introduction

The following embodiments generally relate to external elastic media (EEM)-related analytic data representation of intravascular images. In one embodiment, a non-transitory computer-readable medium is provided that stores program instructions. When executed by one or more processors, the program instructions cause the one or more processors to perform functions comprising: calculating EEM-related analytic data from each of a plurality of cross-sectional intravascular images; and causing a display of: a longitudinal view of the plurality of cross-sectional intravascular images at a selected cutting plane angle; and a longitudinal view of a visualization of the EEM-related analytic data that was calculated from each of the plurality of cross-sectional intravascular images.

In another embodiment, an imaging system is provided comprising: one or more processors; a non-transitory computer-readable medium; and program instructions stored on the non-transitory computer-readable medium. When executed by the one or more processors, the program instructions cause the one or more processors to perform functions comprising: calculating EEM-related analytic data from each of a plurality of cross-sectional intravascular images; and causing a display of: a longitudinal view of the plurality of cross-sectional intravascular images at a selected cutting plane angle; and a longitudinal view of a visualization of the EEM-related analytic data that was calculated from each of the plurality of cross-sectional intravascular images.

In yet another embodiment, a method is provided comprising: calculating EEM-related analytic data from each of a plurality of cross-sectional intravascular images; and causing a display of: a longitudinal view of the plurality of cross-sectional intravascular images at a selected cutting plane angle; and a longitudinal view of a visualization of the EEM-related analytic data that was calculated from each of the plurality of cross-sectional intravascular images.

Other embodiments are possible, and each of the embodiments can be used alone or together in combination.

### Example Ultrasound Imaging System

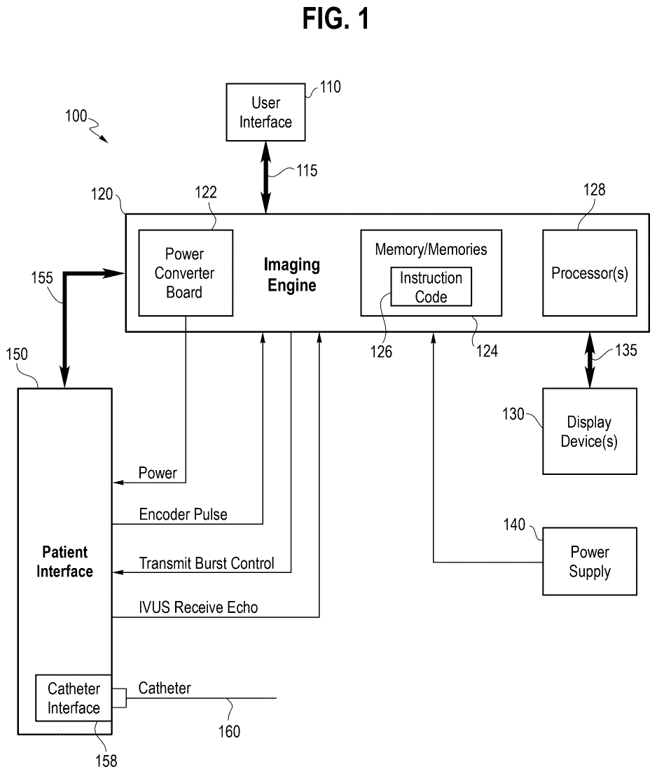

Turning now to the drawings, FIG. 1 is a block diagram of an example medical diagnostic ultrasound imaging system 100 of an embodiment. It should be understood that while an ultrasound system is being used to illustrate this example, these embodiments can be used with other image modalities, e.g. with Optical Coherence Tomography (OCT). As such, the claims should be not limited to ultrasound unless expressly recited therein. As shown in FIG. 1, the ultrasound imaging system 100 of this embodiment comprises a user interface 110 that can comprise one or more real or virtual user input elements (e.g., a keyboard, a mouse, a touch screen, button(s), knob(s), switch(es), a microphone for voice input, etc.) that a user can use to interact with the ultrasound imaging system 100. In this example, the user interface 110 is in communication with an imaging engine 120 via a communication channel 115 (e.g., a Universal Serial Bus (USB) cable). In general, the components shown in FIG. 1 can be in communication with each other using any suitable wired or wireless communication channel.

The imaging engine 120 comprises a power converter board 122, a non-transitory computer-readable medium (i.e., one or more memories, such as RAM, a flash drive, a hard drive, etc.) 124 storing computer-readable program/instruction code 126, and one or more processors 128 configured to execute the computer-readable program/instruction code 126 to perform some or all of the functions described herein and, optionally, other functions. In other embodiments, a pure-hardware implementation (e.g., using logic gates, switches, an application specific integrated circuit (ASIC), etc.) is used. Also, “means” for performing a function can be implemented with one or more processors executing computer-readable program instructions and/or exclusively in hardware.

The imaging engine 120 in this example is in communication with one or more display devices 130 via another communication channel 135. The imaging engine 120 can display generated ultrasound images and other information on the display device(s) 130. The imaging engine 120 receives power from a power supply 140, and the power converter board 122 in the imaging engine 120 provides power to a patient interface module 150 (another communication channel 155 can couple the imaging engine 120 and patient interface module 150). The patient interface module 150 comprises a catheter interface 158 which is electrically and mechanically coupled with a catheter 160. (As used herein, “coupled with” can mean directly coupled with or indirectly coupled with through one or more components, which may or may not be described herein.) The distal end of the catheter 160 in this example comprises a radial ultrasound transducer (not shown) with a mechanically-rotating imaging core that can generate ultrasound images throughout its 360-degree rotation or at selected intervals thereof. The patient interface 150 can also comprises processor(s) and memory/memories (not shown) with instruction code executable by those processors to control the operation of the patient interface 150.

In operation, the patient interface 150 sends an encoder pulse to the imaging engine 120, and the imaging engine 120 sends a transmit burst control signal to the patient interface 150. In response, the patient interface 150 causes a transmit waveform to be transmitted from the ultrasound transducer in the catheter 160. The transducer emits an ultrasonic pressure field to insonify patient tissue (e.g., the coronary artery). Some ultrasonic energy is backscattered and received by the transducer in the catheter 160 as receive echo data. The patient interface 150 sends the receive echo data to the imaging engine 120 for processing to generate and display ultrasound images (and data based on those images) on the display device(s) 130. More information regarding example processing and image generation techniques can be found in U.S. Pat. No. 10,987,086, which is hereby incorporated by reference.

It is important to note that the ultrasound imaging system discussed above is merely an example and that other configurations and types of ultrasound systems can be used. Accordingly, the details presented herein should not be read into the claims unless expressly recited therein.

It is also important to note that some of the embodiments described herein can be implemented in a computing device (e.g., a personal computer (PC), a tablet, etc.) that is separate from an ultrasound imaging system. For example, a computing device can receive images generated by the ultrasound imaging system and process those images as described herein. So, the one or more processors and the non-transitory computer-readable medium storing program instructions for execution by the processor(s) to perform the longitudinal analytic data representation can be contained in a device/system that does not have components for the actual generation of those images. Further, depending on the context, the phrase “obtaining an ultrasound image” (or variations thereof) can mean receiving/retrieving an ultrasound image (e.g., from a storage device, via a network, via an over-the-air transmission, etc.) or can mean actually acquiring the ultrasound image.

### Example Intravascular Ultrasound Image (IVUS) Procedure

The ultrasound imaging system 100 can be used to perform an intravascular ultrasound image (IVUS) procedure to generate ultrasound images of the inside of a blood vessel (e.g., a coronary artery). Such images (and data generated from the images) can show the degree of narrowing (stenosis) of the blood vessel, which can be helpful in assessing coronary artery disease and guiding coronary interventions (e.g., the placement of a bare-metal or a drug-eluting stent, or performing an angioplasty procedure).

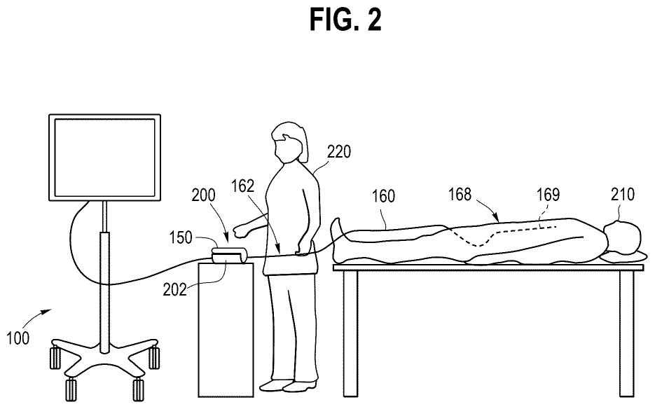

With reference to FIG. 2, during the procedure, a doctor 220 inserts the catheter 160 into the patient 210 (e.g., via the femoral artery) and guides the catheter 160 to an area of interest in the blood vessel of the patient 210. The broken line in FIG. 2 represents the portion of the catheter 160 inside the patient 210. The catheter 160 comprises a radial ultrasound transducer probe 169 at the distal end 168 of the catheter 160. The radial ultrasound transducer probe 169 transmits a sound wave into the blood vessel in a 360-degree fashion and receives echo reflections from the blood vessel. The imaging engine 120 creates an ultrasound image from those reflections, which represents a cross-sectional image of the blood vessel at a given location in the blood vessel.

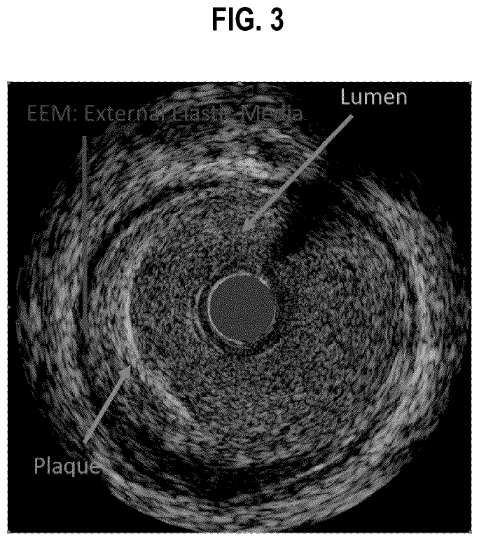

FIG. 3 is an example cross-sectional ultrasound image. As shown in FIG. 3, the cross-sectional ultrasound image shows the lumen of the blood vessel (which is the region where blood flows) and the blood vessel wall (which is sometimes referred to herein as external elastic media (EEM)). The cross-sectional ultrasound image in FIG. 3 shows plaque near the vessel wall, which narrows the size of the lumen and restricts blood flow. The severity of the narrowing of the lumen (stenosis) is an important factor in assessing the extent of coronary artery disease and treatment options. To measure the narrowing, the doctor can use the user interface 110 to manually draw an outline around the outer boundary of the lumen and the inner boundary of the vessel wall. Such manual segmentation can be tedious and time-consuming, and semi- or fully-automatic segmentation methods can be used. Examples of automated segmentation methods are discussed in U.S. Pat. No. 10,275,881, which is hereby incorporated by reference.

As noted above, this cross-sectional image is at one specific location in the blood vessel, and the doctor may need to view cross-sectional images at other locations in the blood vessel to diagnose and treat the patient. As such, in this procedure, the catheter 160 can be moved along the blood vessel, with the radial ultrasound transducer probe 169 transmitting sound waves and receiving echo signals along the way to generate a series of cross-sectional ultrasound images corresponding to a plurality of different locations in the blood vessel. With reference to FIG. 2, to do this, a translation device 200 can translate the ultrasound transducer probe 169 of the catheter 160 using a linear translation system (LTS) 202, which can be mechanically engaged with the catheter 160 and configured to translate the catheter 160 a controlled distance within the patient 210 during a translation operation (e.g., a pullback or push-forward operation). In some embodiments, the LTS 202 can translate the catheter 160 as well as translate the ultrasound transducer probe 169 with respect to the catheter 160. As the ultrasound transducer probe 169 is translated, it generates cross-sectional image data at various longitudinal locations. For example, the imaging engine 120 can generate 40-60 ultrasound images per second and store one frame per every millimeter (or some other distance) that the catheter 160 moves along the blood vessel.

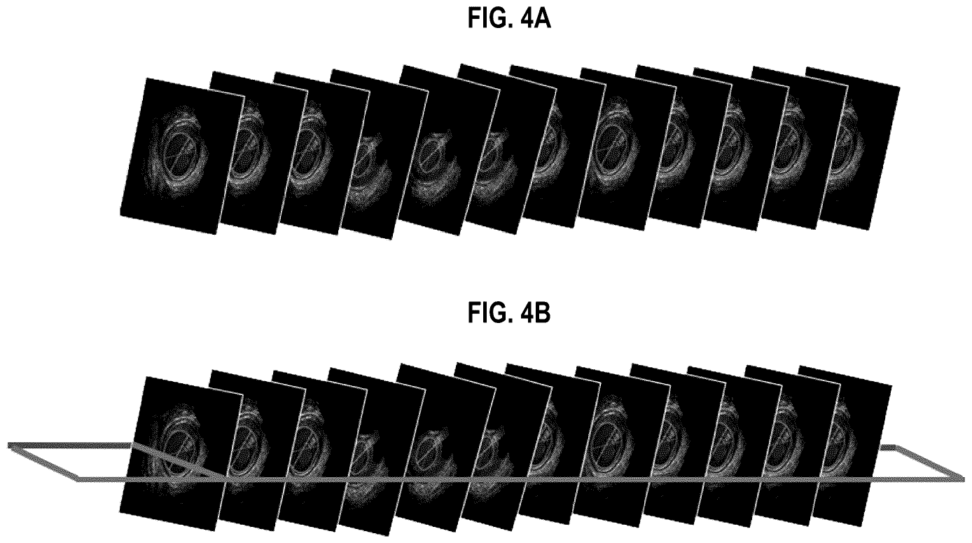

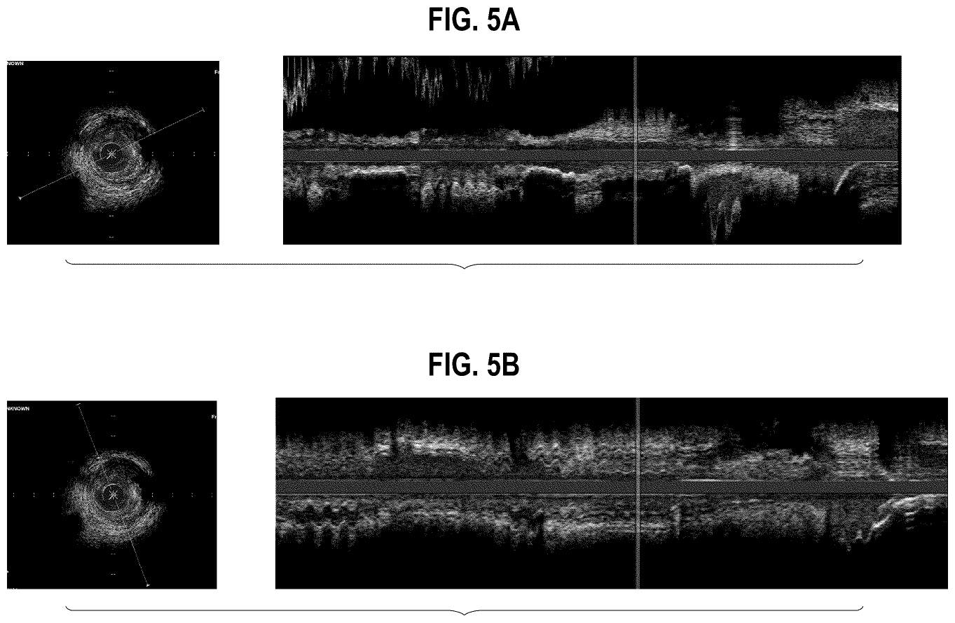

The result of this process is a plurality (or “stack”) of cross-sectional intravascular ultrasound images (see FIG. 4A). To help the doctor visualize the information in all of the images, the imaging engine 120 can compile the cross-sectional image data at various longitudinal locations and generate and display, on the display device 130, a longitudinal view constructed from the stack of cross-sectional intravascular ultrasound images. An example longitudinal view is shown in FIG. 5A, where a rectilinear line 500 (depicted in the left image of FIG. 5A) shows a selected location in the stack, and the cross-sectional ultrasound image at the selected location is displayed in longitudinal view (right image of FIG. 5A). To help the doctor interpret the longitudinal view, the imaging engine 120 can automatically segment the stack of ultrasound images to identify the boundaries of the lumen and vessel wall in each of the images and place lines around those boundaries in the longitudinal view. It is almost impossible to manually annotate an entire patient image data set generated at 60 frames per second. However, with the development of computer algorithms, including artificial intelligence (AI), patient image data of, for example, 2,000-3,000 frames can be automatically annotated with the lumen and EEM border.

From this longitudinal view, the doctor can see the lumen, plaque, and vessel wall locations along the length of the blood vessel that is imaged. From this view, the doctor can see areas in the blood vessel that have a relatively-large build-up of plaque compared to other areas in the blood vessel. The longitudinal view can provide important information to the doctor for deciding a stent landing zone, as well as the size and length of the stent. However, this longitudinal view can be deceiving because the data that is visualized is just a subset of the overall data. More specifically, the longitudinal view that is displayed in FIG. 5A is based on a specific angle (or “cutting plane”) in the stack of images that is shown in FIG. 4B as well as illustrated by the diagonal line through the cross-sectional ultrasound image next to the longitudinal view in FIG. 5A. As shown in FIG. 5B, when a different cutting plane is chosen in the cross-sectional ultrasound image (as indicated by the rectilinear line in the cross-sectional ultrasound image being in a different location as compared in FIG. 5A), a different longitudinal view is shown because the lumen, plaque, and vessel wall are not symmetrical about the ultrasound transducer probe 169.

The result of all this is that a location that looks like it has relatively-little plaque build-up in the cutting angle represented in the longitudinal view of FIG. 5A may have a relatively-large amount of plaque build-up in the cutting angle represented in the longitudinal view of FIG. 5B, and vice versa. So, to get a complete picture of the health of the blood vessel and decide upon the following treatment to be pursued, the doctor may need to rotate the cutting plane for a given location in the blood vessel and then repeat that process for several other locations in the blood vessel. Not only can this be a tedious task, but it may be difficult for the doctor to retain and process the large amount of data that he is viewing. As a result, a doctor may rely on only a small handful of manually-selected frames, thus relying on only a part of all of the information that is available. Even in so limiting the data set, the doctor has to remember and process the information in his head to diagnose and treat patients accordingly. Further, there can be a great difference in expertise depending on the experience, training, and how many various cases were encountered by individual doctors. As a result, it can take long time for new doctors to train, and there may be concerns about trial and error in the process.

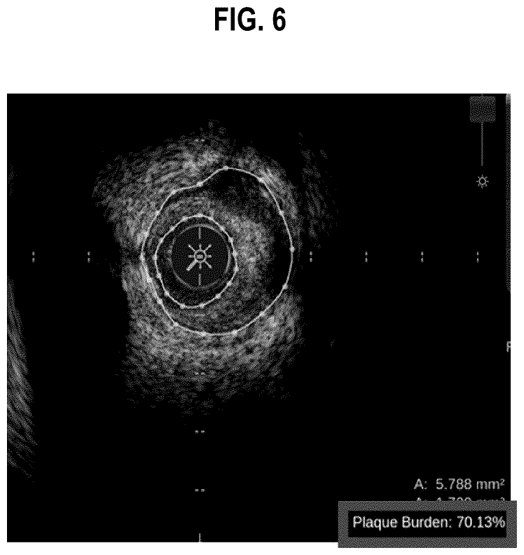

This problem can occur quantitively as well with EEM-related analytic data (e.g., EEM area, EEM average diameter, and/or plaque burden). For example, plaque burden can be important information for the doctor in determining the extent of plaque buildup in the coronary artery and can provide important information to the doctor for deciding a stent landing zone, as well as size and length of the stent. FIG. 6 shows a display of an example plaque burden computation based on the following equation after the lumen and EEM areas have been automatically calculated:

$$Plaque Burden = 100 * \frac{EEM Area - Lumen Area}{EEM Area}$$

In this calculation, the lumen and EEM areas are calculated based on measurements taken from a specific cutting angle of a cross-sectional ultrasound image. For the reasons mentioned above, these measurements can vary depending on the cutting angle. So, basing the lumen and EEM areas on measurements taken along one particular cutting plane may not represent the actual areas. As such, the calculated plaque burden may not be accurate. The same problem can occur in other analytic data calculations that are dependent on the cutting plane angle, such as, but not limited to, minimum lumen area (MLA) and average lumen diameter.

### Example Embodiment of EEM-Related Analytic Data Representation of Intravascular Images

To address this issue, in one embodiment, EEM-related analytic data is calculated from each of a plurality of cross-sectional intravascular images. A longitudinal view of the plurality of cross-sectional intravascular images at a selected cutting plane angle is displayed, as well as a longitudinal view of a visualization of the EEM-related analytic data that was calculated from each of the plurality of cross-sectional intravascular images. The EEM-related analytic data can be, for example, EEM area, EEM average diameter, and/or plaque burden.

Additionally, instead of calculating EEM-related analytic data based on a single cutting plane angle of each cross-sectional ultrasound image in a stack, the processor(s) 128 can calculate the EEM-related analytic data based on a plurality of cutting plane angles. By calculating the EEM-related analytic data based on more than one cutting plane angles (e.g., on all of the cutting plane angles or some suitable number more than one), the resulting data is more representative of the actual imaged anatomy. This avoids the “deceptive data problem” noted above when EEM-related analytic data is based on measurements taken at just one cutting plane angle, which may not reflect the true relevant dimension (e.g., by the minimum and maximum lumen diameter and dividing by two may not be as accurate as measuring the lumen diameter per angle across the entire 180 degrees contour or some angular thereover more than a single angle). This also avoids the need for the doctor to view a plurality of cutting plane angles for each cross-sectional ultrasound image to get desired information.

The EEM-related analytic data calculated from this more-robust data set can be displayed in an intuitive visual format (in addition to or instead of the longitudinal view discussed above). The intuitive display can provide a large amount of information to a doctor in an easy-to-understand way to help the doctor promptly decide a treatment plan and/or review treatment results during a post-stent review or a post angioplasty treatment.

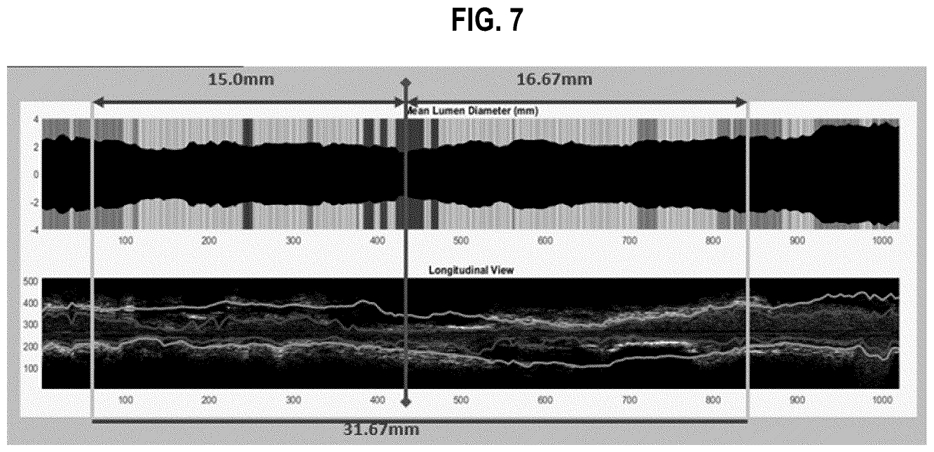

Turning again to the drawings, FIG. 7 is an example visual representation of EEM-related analytic data of an embodiment. While FIG. 7 shows the EEM-related analytic data representation above the display of a longitudinal view, the EEM-related analytic data representation can be displayed in any suitable location with or without the display of the longitudinal view. In this example, the longitudinal view at the bottom portion of FIG. 7 is similar to what was described above but with red lines showing the lumen boundary and the green lines showing the EEM boundary (any suitable indication can be used for these representations). As discussed above, the boundaries in this longitudinal view are based on the particular cutting plane angle being represented. So, the location of the red and green lines (the lumen and EEM boundaries) can change as the cutting plane angle changes.

The top portion of FIG. 7 is the longitudinal EEM-related data representation. In this example, the EEM-related analytic data being represented is average (mean) lumen diameter and the plaque burden. As noted above, these calculations are based on a plurality of (e.g., all) cutting plane angles of each cross-sectional ultrasound image in the stack along the imaged blood vessel. As such, unlike the longitudinal view at the bottom of FIG. 7 that will change as the selected cutting angle changes, the visual representation of the average (mean) lumen diameter and the plaque burden will not, as those values are calculated across the plurality of the cutting plane angles and, hence, will not change based on the view of a specific cutting plane angle that was already considered in the calculation.

Describing the visual in FIG. 7 in more detail now, FIG. 7 shows the average lumen diameter at each location in the blood vessel as a series of vertical bars along a millimeter scale. The black color in the middle represents the open space through which blood flows in the vessel. As noted above, because the average lumen diameter was calculated from a plurality of cutting plane angles in the stack of cross-sectional ultrasound images, these vertical bars (and, hence, the black space in the middle) are vertically-mirrored about a horizonal line along the span of the recording at the 0 mark on the scale. Accordingly, the display of the average lumen diameter does not change when the longitudinal view changes after a different cutting plane angle is selected.

Each vertical line is also color coded to represent the plaque burden at each location in the blood vessel. In this example, green represents a plaque burden less than 50%, yellow represents a plaque burden between 50% and 70%, and red represents a plaque burden over 70%. It should be noted that while color coding is used in this example, other visual indicia can be used, such as, but not limited to, different degrees of grayscale, different hatchings, different line weights, text (e.g., with codes representing different percentages or the display of the percentages themselves), etc. Again, because the processor(s) 128 calculates the plaque burden at a plurality of cutting plane angles and not at just a single cutting plane angle, the visualization of the plaque burden does not change when the longitudinal view changes after a different cutting plane angle is selected.

In summary, the bottom portion of the display of this example embodiment shows the longitudinal view at a certain cutting plane angle, and the top portion of the display shows the longitudinal view with the lumen and plaque burden indicators superimposed. Additionally, the minimum/maximum area or diameter for both the lumen and EEM border (the yellow and purple vertical bar in FIG. 7) can provide useful information. The minimum area/diameter can be the high-potential region for treatment while the maximum area/diameter regions can be the high-potential regions for stent landing zones.

It should be noted that while FIG. 7 shows the EEM-related analytic data being represented as average lumen diameter and the plaque burden, other analytic data can be displayed in addition to or instead of one or both of these items. Other analytic data can include, but is not limited to, minimum lumen area (MLA) and EEM area.

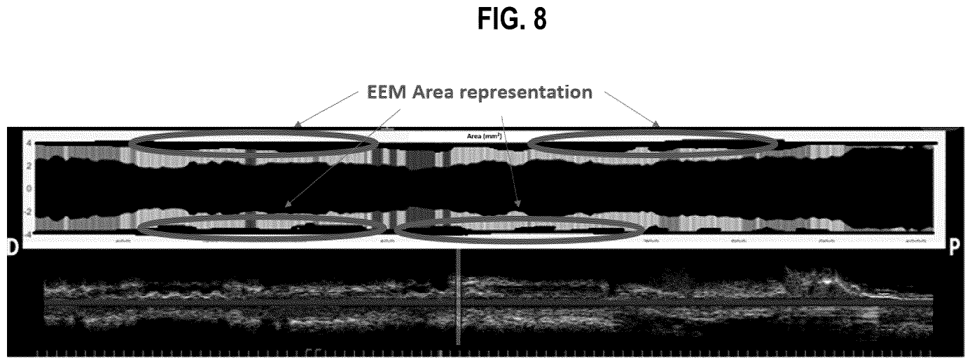

FIG. 8 shows the representation of EEM area in addition to the representation of average lumen diameter and plaque burden. The average EEM diameter (or area) of the blood vessel is displayed by cutting more black/empty space out of the shape described above. The lumen and EEM annotations allow the physician to see the shape of the cross-section of the vessel at a given angle, superimposed over the longitudinal view of the vessel. The physician can adjust the cross-section angle of the vessel in real-time. This can provide an accurate representation of the extent of plaque buildup and EEM dimensions, which can be important for proper decision-making during interventional procedures. More specifically, the difference in EEM dimensions can indicate greater plaque burden. That is, the technical benefit of representing the EEM area “border” beyond just plaque burden is that the EEM boundary represents the actual blood vessel wall, whereas the lumen represents just the blood flow region within the blood vessel. Without the EEM border, it can be difficult, if not impossible, to distinguish between, for example, two vessels with the same lumen contour but where one is much larger and has much more plaque than the other. With the EEM border visualized, the physician can get a full visual representation of the area being worked on. When physicians decide the stent size, the size (diameter, area) of actual blood vessel wall (EEM) can be an important factor.

As mentioned above, the information conveyed by the visualization of EEM-related analytic data in this embodiment can be useful to a physician. For example, the data representation can convey the average diameter of the lumen along the blood vessel, which can inform the physician of the location of narrow regions with restricted blood flow, which can be areas of interest during the stenting procedure. As another example, the data representation can convey the average degree of plaque burden across the vessel. This can inform the physician of the location of viable landing zones for the stent (regions with low plaque or healthy regions with no plaque at all are typically necessary for the stent to properly anchor to the vessel). As yet another example, the average shape of plaque across the vessel can be conveyed. Beyond just color indicators showing plaque burden, the EEM cutout discussed above can display the average distance between the lumen and EEM along the vessel. Additionally, the actual shape of the lumen and EEM at a given cross-section angle can be conveyed. Because the information discussed above are representations of average diameter/area, the cross-sectional view of the lumen and EEM can be used to see the actual shape of the vessel along the recording.

As can be seen by the above examples, there are several advantages associated with these embodiments. For example, visually representing the plaque burden, lumen, EEM, and average diameter over a longitudinal view allows for quick and easy digestion of information by a physician during a stenting procedure. This can lead to better decision making during interventional procedures and improved patient outcomes. These embodiments can also provide benefits over Optical Coherence Tomography (OCT).

OCT uses a light as an imaging source, which may not be able to penetrate plaque regions. So, while OCT can provide lumen area and average lumen diameter over the longitudinal view, it cannot image the blood vessel wall when there is plaque. Unlike OCT, ultrasound imaging can represent the EEM, so IVUS can provide the plaque burden and EEM area in addition to the minimum lumen area and average lumen diameter. That is, IVUS offers some intrinsic technical advantages versus OCT, such as the possibility to “see” (and thus to “represent”) the EEM border, which means the possibility of calculating the EEM area as well as the plaque area (plaque area=EEM area−lumen area).

Additionally, the resulting graphical representation of these embodiments is an improved user interface over prior user interfaces that displayed the longitudinal view.

Further, these embodiments use extensive data manipulation in both the automatic segmentation and the calculated analytic data at a plurality of cutting angles in each cross-sectional ultrasound image. Also, these embodiments can be used to identifying a desired location for stent placement based on the calculated data, which is a technical improvement that provides a practical application.

### Example Workflow

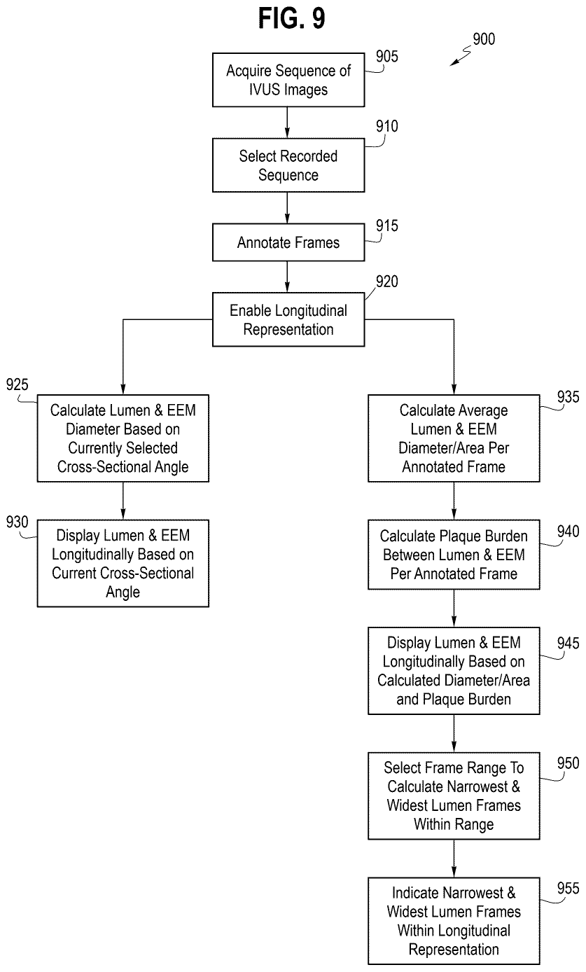

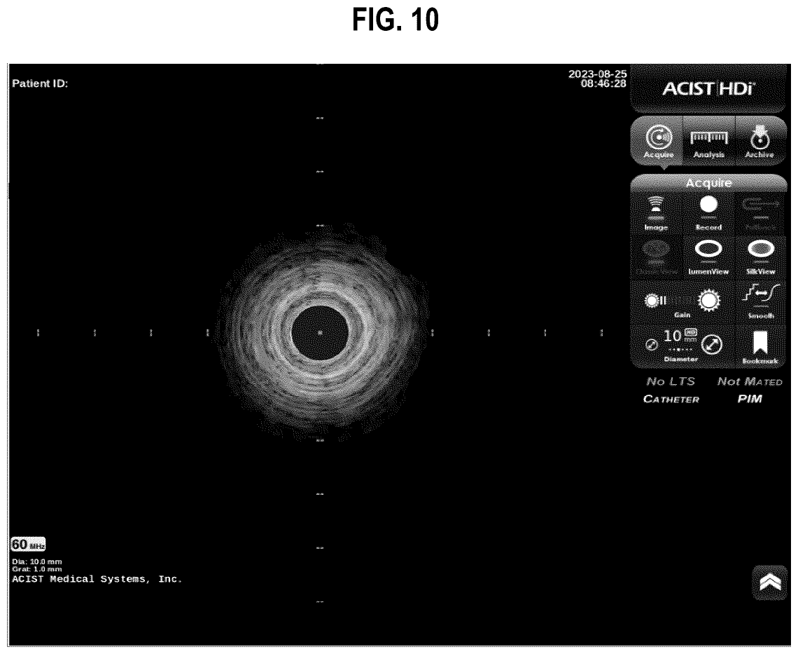

The following paragraphs describe an example workflow that will be illustrated in conjunction with the flow chart 900 in FIG. 9 and the screen shots in FIGS. 10-18. It should be understood that this is merely an example and that other workflows can be used. Also, while these paragraphs use terminology associated with the specific ultrasound system used in this example, it should be understood that the concepts discussed below can be adapted to other suitable ultrasound systems. As such, the specifics provided below are merely intended for illustration and should not be read into the claims. Also, the information used in the calculation of the longitudinal analytic data can be obtained after the segmentation of each cross-sectional frame. Segmentation on frame data can be done using any suitable method and, in one embodiment, it is done with the help of artificial intelligence (e.g., U-Net Image Segmentation). Manual segmentation is also possible.

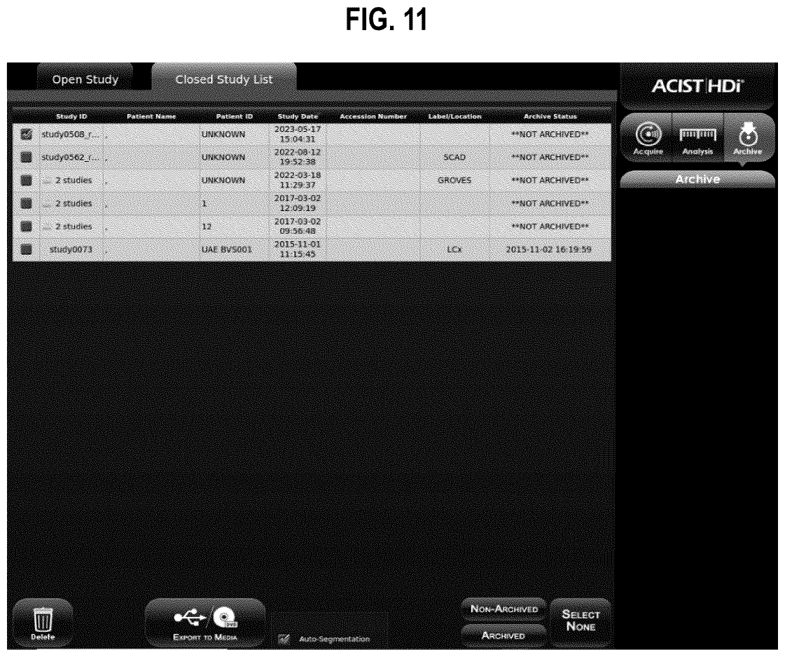

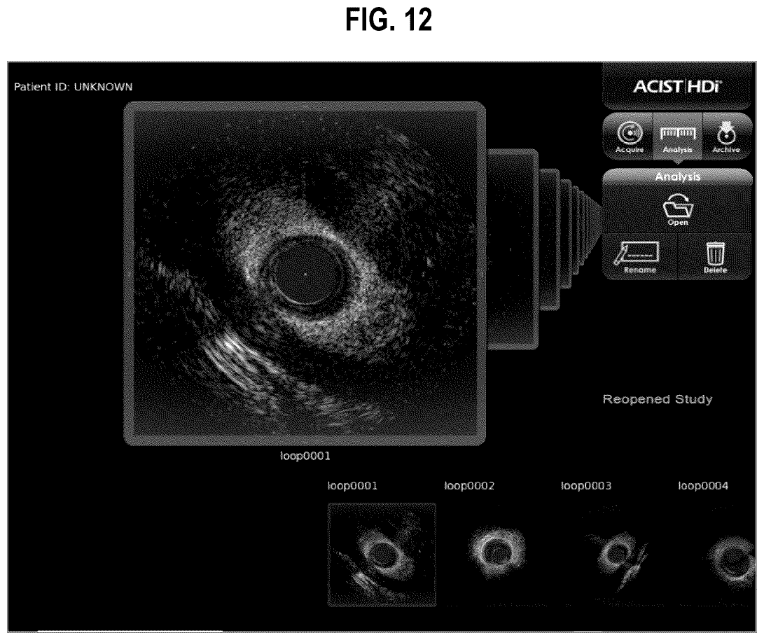

Turning now to FIG. 9, the first step in this example workflow is to acquire a sequence of IVUS images (act 905). FIG. 10 is a screen shot of an acquire view of an embodiment, which shows a “loop” of image data being captured as the IVUS catheter is pulled back from patient artery. As used herein, a ‘loop’ refers to a sequence of IVUS frames captured during catheter pullback. Next, the user selects a recorded sequence (act 910). In this step, after the IVUS loop sequence is captured, the user navigates to the archive view (see FIG. 11) and selects the study associated with the recorded loop. In this example, the screen has a checkbox to enable automatic segmentation (checkbox is shown in the bottom line of the Figure). If checked, the selected loop will be automatically annotated with lumen and EEM boundaries. Otherwise, the loop will be opened directly without any automatic processing. Some studies can have multiple loops (e.g., pre-stent and post-stent pullbacks). As shown in FIG. 12, in that situation, upon loading a study, the user can be given an option to select a loop to view from a series of thumbnail previews.

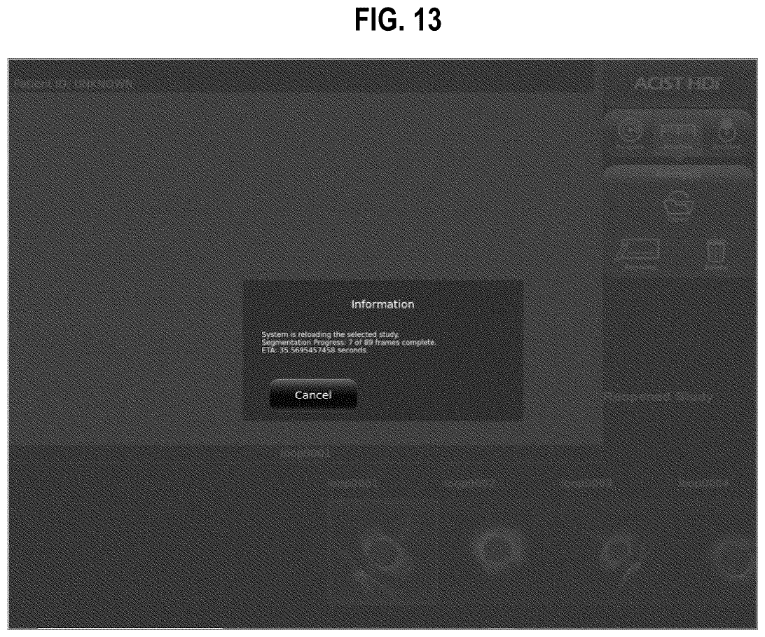

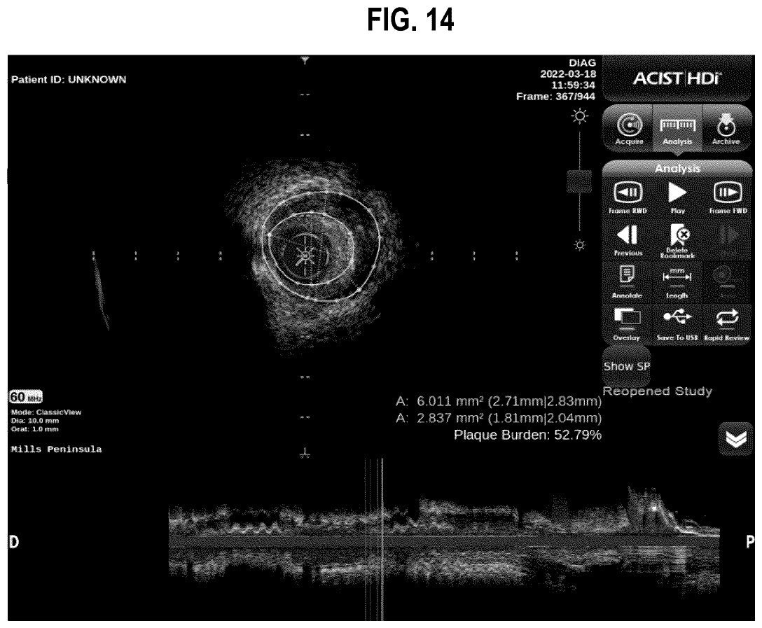

Next, the frames in the loop are annotated (act 915). If the automatic segmentation checkbox (see FIG. 11) was enabled, the processor(s) 128 can automatically segment the selected loop. Upon completion of automatic segmentation, one frame per millimeter will have lumen and EEM annotations in this example. FIG. 13 is a screen shot of an embodiment showing auto segmentation running. If auto segmentation was disabled, the study can be loaded without any annotated frames. The user can create manual lumen and EEM annotations on the loaded study on any desired frame (FIG. 14 is a screen shot of an example manual study annotation). Otherwise, the loaded study will have an automatically-annotated frame every one millimeter. While automatic segmentation/annotation (e.g., using artificial intelligence) may be preferred, manual segmentation/annotation can be used (although much more time consuming and probably on a much smaller set of IVUS images).

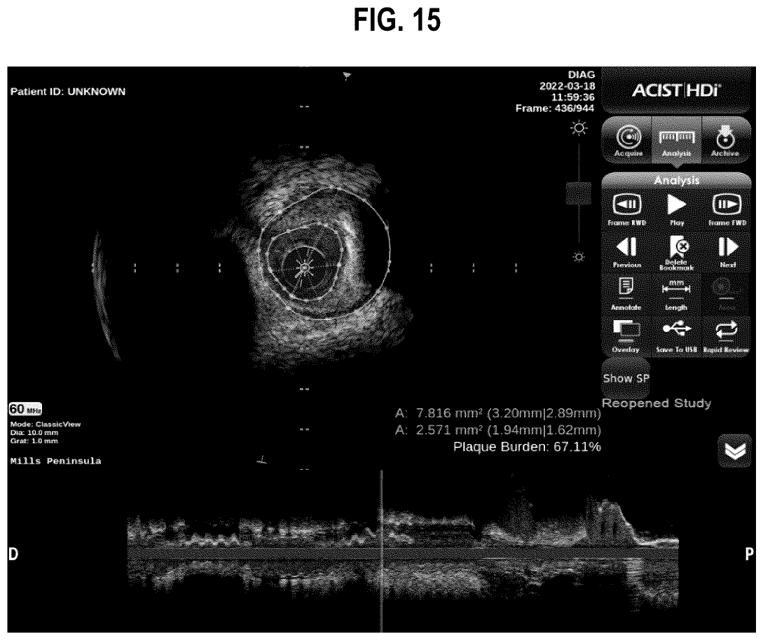

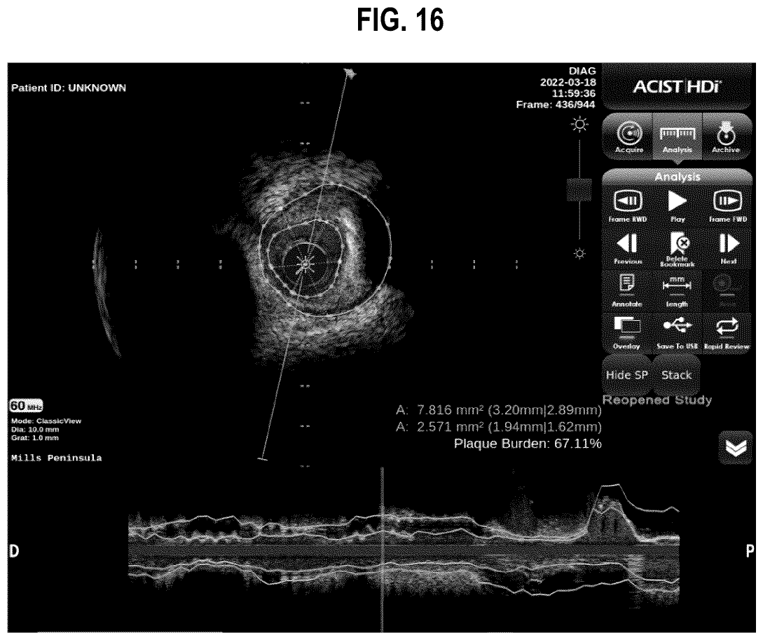

Next, the user enables longitudinal analytic data representation (act 920) In this example, the user does this by selecting the “Show SP” button (“SP” refers to the “Smart Planning” features of this system). FIG. 15 is a screen shot of an embodiment in which a smart planning button in untoggled. In this embodiment, the longitudinal analytic data representation has two modes, which are represented by the two branches of the flow chart 900 in FIG. 9. The first mode is the “overlay” mode. In this mode, the processor(s) 128 can calculate the lumen and EEM diameters of each annotation (automatic or manual) in the loop given the selected cross-sectional angle (act 925) and superimpose this information over the longitudinal loop view (act 930), which is illustrated at the bottom of the screen in FIG. 16. When the angle is adjusted, this representation will be adjusted to reflect the new angle. This view can be used to quickly convey the shape of the lumen and EEM of the artery at a given angle to the user.

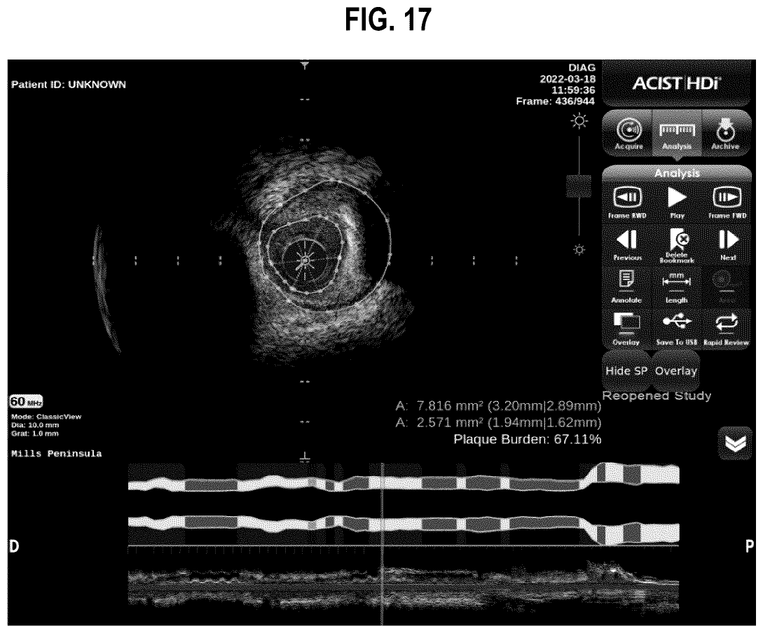

The second view mode for the longitudinal analytic data representation is the “stack” mode. In this mode, the processor(s) 128 calculates the average lumen and EEM diameters for each (automatic or manual) annotated frame (act 935), as well as the plaque burdens of each frame based on these diameter calculations (act 940). This information is displayed alongside the longitudinal loop view (act 945). As shown in FIG. 17, in this example, different colors indicate different plaque burden along the loop, with red representing >70% plaque burden, yellow representing 70%-50% plaque burden, and green representing <50% plaque burden. This view can be used to quickly convey to the user the average lumen and EEM shape of the artery along the loop, as well as plaque burden. As noted above, different visual indicia other than colors can be used. Also, the color representation shown in this figure is different from the color representation shown in FIG. 7, highlighting the fact that any suitable visible representation can be used.

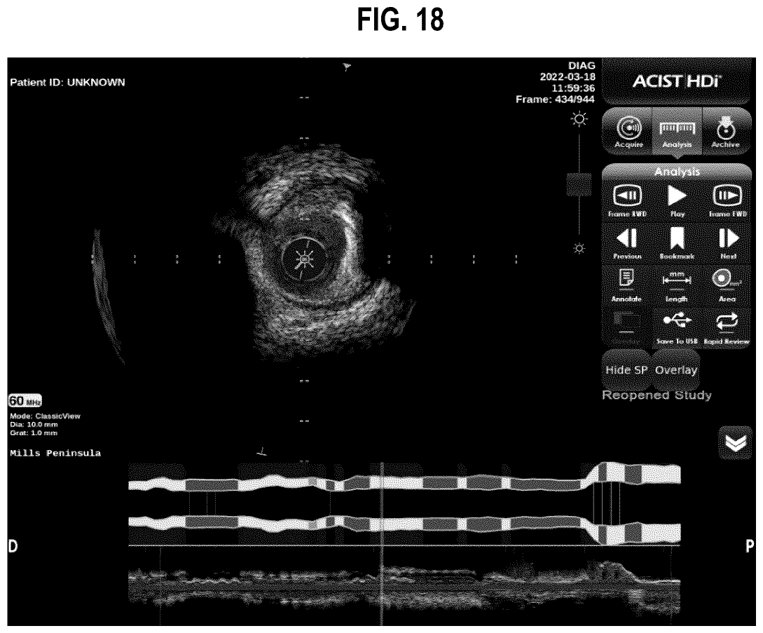

Additionally, in this mode, the user can select a range of frames (start frame, end frame) to calculate the top four (or any suitable number) widest and narrowest lumen frames (act 950), which will then be indicated on the longitudinal view (act 955) (see FIG. 18). This “stack” feature can be useful to easily indicate the narrowest and widest regions, which can be used by the doctor in deciding where to implant a stent. The calculate of the narrowest and widest regions can be done by considering measurements taken at a plurality of cutting plane angles.

### CONCLUSION

Various examples of systems, devices, and/or methods are described herein.

Any embodiment, implementation, and/or feature described herein as being an “example” is not necessarily to be construed as preferred or advantageous over any other embodiment, implementation, and/or feature unless stated as such. Thus, other embodiments, implementations, and/or features may be utilized, and other changes may be made without departing from the scope of the subject matter presented herein.

Accordingly, the examples described herein are not meant to be limiting. It will be readily understood that the aspects of the present disclosure, as generally described herein, and illustrated in the figures, can be arranged, substituted, combined, separated, and designed in a wide variety of different configurations.

Further, unless the context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, terms such as “A coupled to B” or “A is mechanically coupled to B” do not require members A and B to be directly coupled to one another. It is understood that various intermediate members may be utilized to “couple” members A and B together.

Moreover, terms such as “substantially” or “about” that may be used herein, are meant that the recited characteristic, parameter, or value need not be achieved exactly but that deviations or variations, including, for example, tolerances, measurement error, measurement accuracy limitations and other factors known to a skill in the art, may occur in amounts that do not preclude the effect the characteristic was intended to provide.

It is intended that the foregoing detailed description be understood as an illustration of selected forms that the invention can take and not as a definition of the invention. It is only the following claims, including all equivalents, that are intended to define the scope of the claimed invention. Finally, it should be noted that any aspect of any of the embodiments described herein can be used alone or in combination with one another.

What is claimed is:

1. A non-transitory computer-readable medium storing program instructions that, when executed by one or more processors, cause the one or more processors to perform functions comprising:
calculating external elastic media (EEM)-related analytic data from each of a plurality of cross-sectional intravascular images; and
causing a display of:
a longitudinal view of the plurality of cross-sectional intravascular images at a selected cutting plane angle; and
a longitudinal view of a visualization of the EEM-related analytic data that was calculated from each of the plurality of cross-sectional intravascular images.

2. The non-transitory computer-readable medium of claim 1, wherein the EEM-related analytic data comprises EEM area.

3. The non-transitory computer-readable medium of claim 2, wherein the EEM area is displayed by cutting space out of a displayed shape.

4. The non-transitory computer-readable medium of claim 1, wherein the EEM-related analytic data comprises EEM average diameter.

5. The non-transitory computer-readable medium of claim 1, wherein the EEM-related analytic data comprises plaque burden.

6. The non-transitory computer-readable medium of claim 5, wherein a plurality of colors are used to visualize different plaque burdens.

7. The non-transitory computer-readable medium of claim 1, wherein changing the selected cutting plane angle changes the longitudinal view of the plurality of cross-sectional intravascular images but does not change the longitudinal view of the visualization of the EEM-related analytic data.

8. The non-transitory computer-readable medium of claim 1, wherein the non-transitory computer-readable medium is located in an imaging system.

9. The non-transitory computer-readable medium of claim 1, wherein the plurality of cross-sectional intravascular images comprises ultrasound images.

10. An imaging system comprising:
one or more processors;
a non-transitory computer-readable medium; and
program instructions stored on the non-transitory computer-readable medium that, when executed by the one or more processors, cause the one or more processors to perform functions comprising:
calculating external elastic media (EEM)-related analytic data from each of a plurality of cross-sectional intravascular images; and
causing a display of:
a longitudinal view of the plurality of cross-sectional intravascular images at a selected cutting plane angle; and
a longitudinal view of a visualization of the EEM-related analytic data that was calculated from each of the plurality of cross-sectional intravascular images.

11. The imaging system of claim 10, wherein the EEM-related analytic data comprises EEM area.

12. The imaging system of claim 10, wherein the EEM-related analytic data comprises EEM average diameter.

13. The imaging system of claim 10, wherein the EEM-related analytic data comprises plaque burden.

14. The imaging system of claim 10, wherein changing the selected cutting plane angle changes the longitudinal view of the plurality of cross-sectional intravascular images but does not change the longitudinal view of the visualization of the EEM-related analytic data.

15. The imaging system of claim 10, wherein the plurality of cross-sectional intravascular images comprises ultrasound images.

16. The imaging system of claim 10, wherein the imaging system comprises an ultrasound imaging system.

17. A method comprising:
calculating external elastic media (EEM)-related analytic data from each of a plurality of cross-sectional intravascular images; and
causing a display of:
a longitudinal view of the plurality of cross-sectional intravascular images at a selected cutting plane angle; and
a longitudinal view of a visualization of the EEM-related analytic data that was calculated from each of the plurality of cross-sectional intravascular images.

18. The method of claim 17, wherein the EEM-related analytic data comprises EEM area.

19. The method of claim 17, wherein the EEM-related analytic data comprises EEM average diameter.

20. The method of claim 17, wherein the EEM-related analytic data comprises plaque burden.
